(19) United States
(12) Patent Application Publication
(10) Pub. No.: US 2025/0387630 A1
(43) Pub. Date: Dec. 25, 2025

(54) COMPLIANT ELECTRODE FOR IMPLANTABLE MEDICAL DEVICE

(71) Applicant: Medtronic, Inc., Minneapolis, MN (US)

(72) Inventors: Scott G. Terry, Lawrence, KS (US); Kevin T. O'Brien, Arden Hills, MN (US); Troy T. White, Maple Grove, MN (US); Jonathan E. Baxter, Fridley, MN (US); Alyeska P. Dellinger, Minneapolis, MN (US)

(21) Appl. No.: 19/240,466

(22) Filed: Jun. 17, 2025

Related U.S. Application Data
(60) Provisional application No. 63/661,745, filed on Jun. 19, 2024.

(51) Int. Cl. A61N 1/375; A61N 1/05; A61N 1/362

(52) U.S. Cl. CPC A61N 1/3756; A61N 1/0573; A61N 1/362; A61N 1/37512

(57) ABSTRACT

A device comprising: an elongated housing extending from a proximal end to a distal end along a longitudinal axis, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode extends wholly around the longitudinal axis, wherein the second electrode is configured to at least partially deform to contact wall tissue of the chamber without penetrating the wall tissue.

### RELATED APPLICATIONS

This application claims the benefit of U.S. Provisional Application Ser. No. 63/661,745, filed Jun. 19, 2024, the entire contents of each of which are incorporated herein by reference.

### TECHNICAL FIELD

The disclosure relates to medical devices, and more particularly to fixation mechanisms of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to implantable medical devices (IMDs) configured to sense and deliver electrical signals to tissue of a patient via a plurality of electrodes at or near a distal end of an elongated housing of the IMD. More particularly, this disclosure is directed to IMDs with a compliant electrode disposed at or near the distal end of the elongated housing.

In some examples, a single IMD is implanted in one chamber of a heart of the patient and is able to sense in and/or deliver cardiac pacing to more than one chamber, which may avoid the need for a leaded device or multiple smaller devices to provide such functionality, which may reduce the amount of material implanted within the patient. In some examples, such an implantable medical device includes a distal electrode that is configured to penetrate through wall tissue of the heart chamber in which the device is implanted, and into wall tissue of another heart chamber. In addition to the distal electrode, the device includes a reference electrode and one or more proximal electrodes configured to contact the wall tissue of the heart chamber. The distal electrode may be a helix configured to penetrate tissue of the patient. The distal electrode may be configured to sense in and/or deliver cardiac pacing to one chamber of the heart and the one or more proximal electrodes may be configured to sense in and/or deliver cardiac pacing to another separate chamber of the heart.

In some examples, depending on patient physiology at or near a target location, the clinician may need to implant the IMD at different angles and/or at different depths. For example, the clinician may need to implant the IMD within the chamber at a target location which may not have any substantially flat surfaces, which may require the clinician to implant the IMD into the target location at an angle. Implantation of the IMD at an angle may increase a risk of dislodgement of the IMD from the tissue and/or causing the distal electrode of the IMD to be implanted into the tissue at an insufficient depth.

This disclosure describes a compliant electrode disposed at or around the distal end of the elongated body of the IMD. At least a portion of the compliant electrode may elastically deform (e.g., compress) as the IMD is implanted into tissue at an angle, thereby increasing a range of possible implantation angles for the IMD. For example, the compliant electrode may allow the IMD to be implanted into the tissue orthogonally or at a range of angles offset from the tissue surface. The compliant electrode may also compress as the clinician advances the distal electrode further into the tissue, thereby allowing for increased control of the depth of the distal electrode within the tissue compared to other IMDs.

In some examples, this disclosure is directed to a device comprising: an elongated housing extending from a proximal end to a distal end along a longitudinal axis, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode extends wholly around the longitudinal axis, wherein the second electrode is configured to at least partially deform to contact wall tissue of the chamber without penetrating the wall tissue.

In some examples, this disclosure is directed to a fixation device comprising: an elongated body extending distally from a distal end of an implantable medical device along a longitudinal axis, the elongated body comprising: a proximal end located at the distal end of the implantable medical device, and a helix extending distally from the proximal end along the longitudinal axis and defining one or more coils, wherein a distal end of the helix is configured to penetrate into tissue of a patient; and a spring disposed on the distal end of the implantable medical device, wherein the spring extends wholly around the longitudinal axis, and wherein the spring is configured to at least partially deform to contact the tissue without penetrating the tissue to inhibit unintended rotation of the elongated body within the tissue.

In some examples, this disclosure is directed to a method comprising: inserting a device into a chamber of a heart, the device comprising: an elongated housing extending from a proximal end to a distal end along a longitudinal axis, a first electrode extending distally from the distal end of the elongated housing, the first electrode comprising an elongated body defining a helix, and a second electrode disposed on the distal end of the elongated housing, wherein the second electrode extends wholly around the longitudinal axis; advancing the first electrode to penetrate wall tissue of the chamber, wherein the second electrode is configured to at least partially deform as the first electrode penetrates the wall tissue; and delivering cardiac pacing from the device to the wall tissue via at least one of the first electrode or the second electrode.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2A is a perspective diagram illustrating an example configuration of the device of FIG. 1 with a compliant electrode.

FIG. 2B is a perspective diagram illustrating an example side view of the device of FIG. 2A.

FIG. 2C is a perspective diagram illustrating an example top view of the device of FIG. 2A.

FIG. 3 is a block diagram illustrating an example configuration of an example device of any of FIGS. 1-2C.

FIG. 4 is a conceptual diagram of an example device of any of FIGS. 1-3 implanted at a target implant site.

FIG. 5A is a conceptual diagram illustrating an orthogonal implantation orientation of an example device of any of FIGS. 1-4.

FIG. 5B is a conceptual diagram illustrating an example angled implantation orientation of an example device of any of FIGS. 1-4.

FIG. 6 is a conceptual diagram of an example compliant electrode of an example device of any of FIGS. 1-6.

FIG. 7 is a flowchart illustrating an example process for sensing a cardiac electrical signal and delivering cardiac pacing therapy to a heart of a patient via an example device of any of FIGS. 1-6.

### DETAILED DESCRIPTION

In general, this disclosure is directed to distal end configurations for implantable medical devices (IMDs). More particularly, this disclosure is directed to IMDs having a compliant electrode disposed at or around a distal end of an elongated housing of the IMD. The compliant electrode may at least partially elastically deform to enable implantation of the IMD into tissue at varying implantation angles and/or to varying implantation depths.

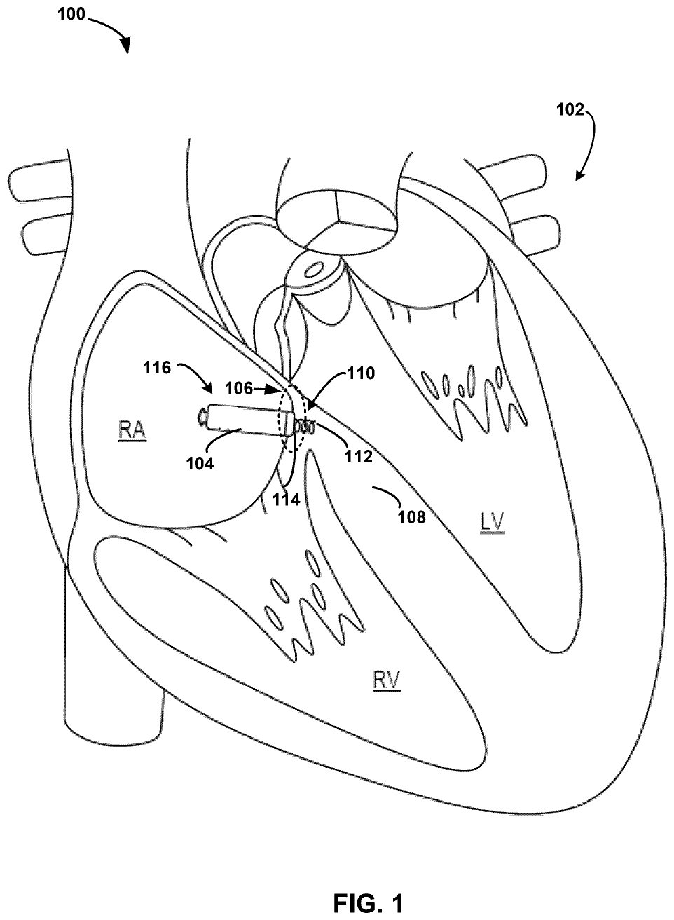

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 of the patient with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve. In some examples, target implant region 106 may be disposed in another position within heart 102, e.g., within a right ventricle (RV) of heart 102.

Device 104 includes a distal end 110 and a proximal end 116. Distal end 110 includes a first electrode 112, and a second electrode 114 (alternatively described herein as “compliant electrode 114”). First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Second electrode 114 may be a compliant electrode and may include, but is not limited to, a wave spring electrode or a coil spring electrode. Second electrode 112 may extend at least a portion or wholly around the outer perimeter of first electrode 112. For example, second electrode 114 may be a wave spring electrode or a coil spring electrode extending wholly around the outer perimeter of first electrode 112.

Second electrode 114 may contact the wall tissue of the first chamber as first electrode 112 penetrates the wall tissue of the first chamber. When device 104 is affixed to the wall tissue of the first chamber, second electrode 114 may at least partially elastically deform (e.g., compress) to place an electrically active region of second electrode 114 in contact with the wall tissue (e.g., without puncturing or penetrating the wall tissue). The compression of second electrode 114 may inhibit unintended rotation of first electrode 112 within the wall tissue. An amount of compression of second electrode 114 may be based on one or more parameters including, but are not limited to, a compliance of second electrode 114, a target implantation depth for first electrode 112, or an implantation angle of device 104 relative to a surface of the wall tissue.

The configuration of electrodes 112 and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 114 may facilitate the delivery of A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. In some examples, first electrode 112 extends into the tissue of heart 102 at region 106 and affix device 104 to the tissue of heart 102.

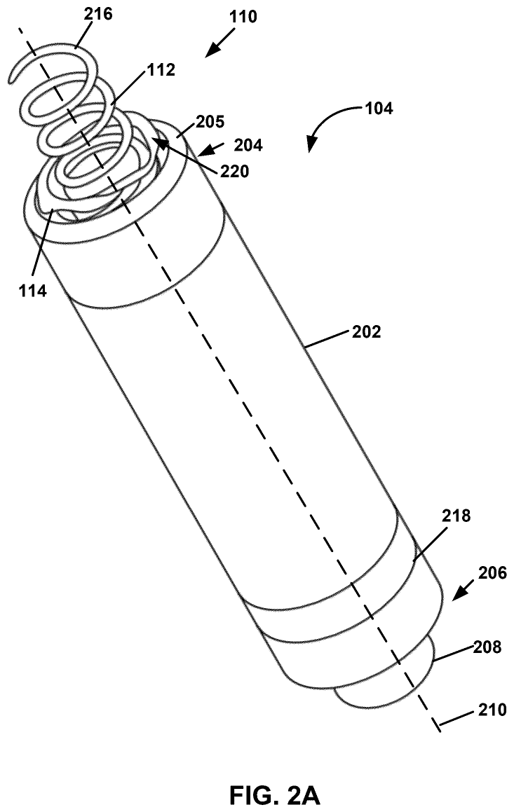

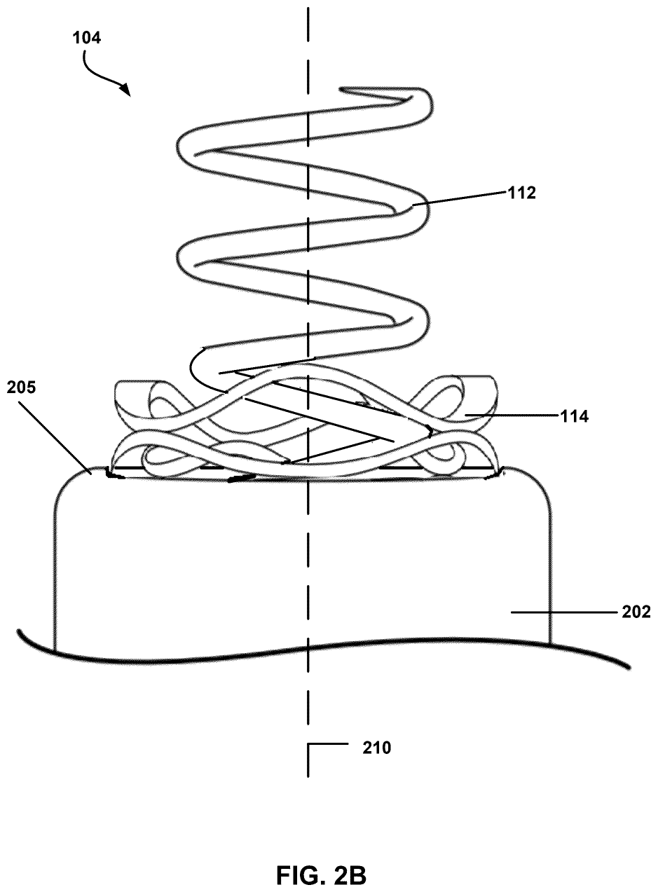

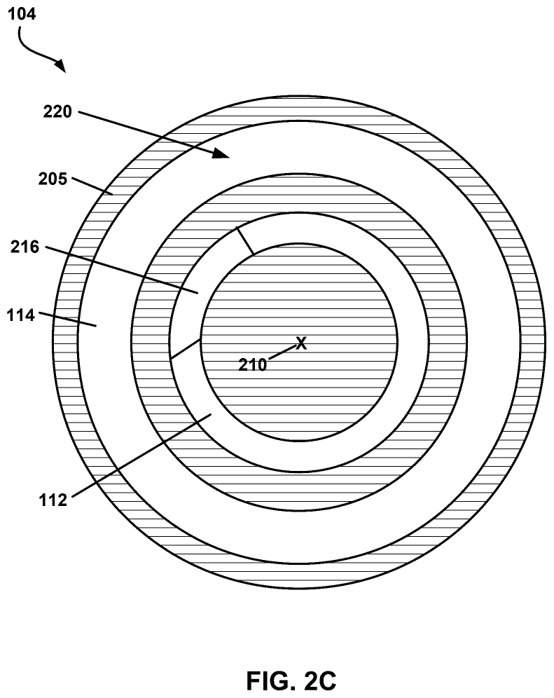

FIG. 2A is a perspective diagram illustrating device 104. Device 104 may include a housing 202 extending from a distal end 204 to a proximal end 206 along longitudinal axis 210. First electrode 112 and second electrode 114 may extend distally along from distal end 204 of housing 202 and along longitudinal axis 210.

Housing 202 may define a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 202 extends between distal end 204 and proximal end 206 along longitudinal axis 210. Housing 202 may be cylindrical or substantially cylindrical but may be other shapes, e.g., prismatic, or other geometric shapes. Housing 202 may include a delivery tool interface member 208, e.g., at proximal end 206, for engaging with a delivery tool during implantation of device 104. At distal end 204, housing 202 may define a face 205 of housing 202. Face 205 may define a distal end major surface. Face 205 may be orthogonal to longitudinal axis 210. In some examples, face 205 is slanted, e.g., face 205 defines a reference plane that is not orthogonal to longitudinal axis 210.

Face 205 may define a distal end of housing 202. Electrodes 112 and 114 my extend distally from face 205 along longitudinal axis 210. In some examples, device 104 includes one or more fixation features (e.g., recesses, protrusions, ramps, meshes, tines, or the like) disposed on face 205. First electrode 112 may define a helical or spiral structure. First electrode 112 may extend distally from face 205 to a distal tip. Second electrode 114 may be disposed substantially fully around longitudinal axis 210, e.g., such that second electrode 114 defines an annular structure. Second electrode 114 may be disposed radially outwards of first electrode 112. In some examples, second electrode 114 is disposed radially inwards of first electrode 112 on face 205.

First electrode 112 may include one or more coatings (e.g., electrically insulative coating(s)) configured to define a first electrically active region 216, or first electrode 112 may otherwise define first electrically active region 216. In some examples, first electrically active region 216 is more proximate to the second, e.g., distal, end of first electrode 112. In the example of FIG. 2A, first electrically active region 216 includes the distal end of electrode 112. Second electrode 114 may include one or more coatings configured to define a second electrically active region 220 on an outer surface of second electrode 114. In some examples, second electrical active region 220 forms a ring around first electrode 112, e.g., to allow for full 360 degrees sensing and/or delivery of electrical signals via second electrode 114. Second electrode 114 may define a compliant shape and may include, but is not limited to, a wave spring electrode or another example spring electrode. Second electrically active region 220 may be separated into two or more electrically active sub-regions, with circumferentially-adjacent electrically active sub-regions being separated by an electrically insulated portion of second electrode 114.

First and second electrodes 112 and 114 may be formed of an electrically conductive material, such as titanium, platinum, iridium, tantalum, stainless steel or alloys thereof. In some examples, second electrode 114 is formed from one or more of Platinum Iridium, a Platinum Iridium-clad alloy (e.g., Platinum Iridium-clad Titanium or Nitinol), Nitinol, or Tantalum Tungsten. First and second electrodes 112 and 114 may be coated with an electrically insulating coating, e.g., a parylene, polyurethane, silicone, epoxy, or other insulating coating, to reduce the electrically conductive active surface area of first and second electrodes 112 and 114, and thereby define first and second electrically active regions 216 and 220. Defining first and second electrically active regions 216 and 220 by covering portions with an insulating coating may increase the electrical impedance of first and second electrodes 112 and 114 and thereby reduce the current delivered during a pacing pulse that captures the cardiac tissue. A lower current drain conserves the power source, e.g., one or more rechargeable or non-rechargeable batteries, of device 104. Similarly, coating the electrically active regions 216 and/or 220 with a low-impedance coating (for example, TiN or IrOx) allows further decrease in electrically active area with commensurate reduction in current drain while preserving the electrodes' ability to electrically capture the cardiac tissue.

Device 104 may include different second electrodes with different compliances based at least in part on the location of target implant region 106 within heart 102. The compliance of second electrode 114 may be represented as a force acting on second electrode 114 per displacement of second electrode 114. The compliance of second electrode may be represented as Newtons per millimeter (e.g., N/mm). The compliance of second electrode 114 may be based on a compliance value of spring electrode 114 when a spring constant of second electrode 114 aligns with a spring constant of tissue at target implant region 106. For example, a manufacturing assembly may determine the compliance of second electrode 114 based on a point of intersection between the spring constant of second electrode 114 and the spring constant of the tissue at target implant region 106, e.g., on a plot illustrating the spring constants in terms of load force (e.g., in N) per unit displacement (e.g., in mm). A manufacturing assembly may select a specific second electrode 114 based on a determination that the compliance of the specific second electrode 114 being less than or equal to a compliance of tissue within target implant region 106. The compliance of second electrode 114 (e.g., in response to a point load on second electrode 114) may be about 0.3 N/mm. The example compliance values of second electrode 114 described above are intended to be non-limiting examples. The range of compliance values for second electrode 114, as described in this disclosure, are not limited to the example values described above.

In some examples, first and second electrodes 112 and 114 include an electrically conducting material coating on first and second electrically active regions 216 and 220 to define the active regions. For example, first and second electrically active regions 216 and 220 may be coated with titanium nitride (TiN). First and second electrodes 112 and 114 may be made of substantially similar material or may be made of different material from one another.

In the example of FIG. 2A, first electrode 112 takes the form of a helix or a coil. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. First electrode 112 may extend from face 205 from a proximal end to a distal end, e.g., defining first electrically active region 216. The proximal end may be a location along first electrode 112 where first electrode 112 extends distally past face 205 of device 104.

In some examples, first electrode 112 includes one or more anti-rotation features. The anti-rotation features may facilitate fixation of first electrode 112 to the tissue. The additional anti-rotation features may include a shape of first electrode 112, dimensions (e.g., outer diameter, pitch, or the like) of first electrode 112, one or more features disposed on an outer surface of first electrode 112, or the like. The shape and/or dimensions of first electrode 112 may include a geometric shape of first electrode 112, a varying diameter configuration of first electrode 112, a varying pitch configuration of first electrode 112, a waveform configuration of first electrode 112, or any combination herein. The one or more anti-rotation features disposed on first electrode 112 may include, but are not limited to, elongated darts, barbs, or tines. In some examples, the anti-rotation features include bumps, ridges, recesses, and/or other texturing disposed on face 205. The one or more anti-rotation features may resist rotation of first electrode 112, e.g., by penetrating the tissue, by increasing the friction between first electrode 112 and the tissue, or the like.

First and second electrodes 112 and 114 may vary in size and shape in order to enhance tissue contact of first and second electrically active regions 216 and 220. For example, first electrodes 112 may have a round cross-section or could be made with a flatter cross-section (e.g., oval or rectangular) based on tissue contact specifications. In some examples, second electrode 114 defines an outer surface that varies in size and shape (e.g., an oval outer surface, an outer surface with a larger diameter, or the like) in order to enhance tissue contact of second electrically active region 220.

The distal end of first electrode 112 can have a conical, hemi-spherical, or slanted edge distal tip with a narrow tip diameter, e.g., less than 1 millimeter (mm), for penetrating into and through tissue layers. The distal end of first electrode can be a sharpened or angular tip or sharpened or beveled edges, but the degree of sharpness may be constrained to avoid a cutting action that could lead to lateral displacement of the distal end of first electrode 112 and undesired tissue trauma. In some examples, first electrode 112 defines a maximum diameter at its base that interfaces with housing distal end 204. In such examples, the outer diameter of the helix defined by first electrode 112 may decrease from housing distal end 204 to the distal end of first electrode 112. In some examples, the diameter of first electrode 112 varies from proximal end 220 to the distal end of first electrode 112. The varying diameter may cause first electrode 112 to resist rotation within the tissue of heart 102.

The outer dimensions of first electrode 112 can be substantially straight and cylindrical, with first electrode 112 being rigid in some examples. First electrode 112 may have flexibility in lateral directions, being non-rigid to allow some flexing with heart motion. In a relaxed state, when not subjected to any external forces, first electrode 112 can be configured to maintain a distance between first electrically active region 216 and housing distal end 204.

Distal end of first electrode 112 can pierce through one or more tissue layers to position first electrically active region 216 within a desired tissue layer, e.g., the ventricular myocardium 108 or interventricular septum. Accordingly, first electrode 112 extends a distance from housing distal end 204 corresponding to the expected pacing site depth and may have a relatively high compressive strength along its longitudinal axis, which may be substantially similar to or coincident with longitudinal axis 210, to resist bending in a lateral or radial direction when a longitudinal, axial, and/or rotational force is applied, e.g., to the proximal end 206 of housing 202 to advance device 104 into the tissue at target implant region 106. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 204 to the distal end of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 210), first electrode 112 retains a straight, linear configuration as shown.

As first electrode 112 enters tissue, second electrode 114 may at least partially compress along longitudinal axis 210. A maximum displacement of second electrode 114 may be based on the compliance of second electrode 114. Device 104 may include second electrode 114 with a target compliance value to allow first electrode 112 to be advanced into the tissue at target implant region 106 to at least a target implantation depth. In such examples, second electrode 114 may not cease compressing until first electrically active region 216 is at the target implantation depth from the surface of the tissue. When second electrode 114 is compressed, second electrically active region 220 may be placed in contact with the surface of the tissue. The compressed second electrode 114 may apply a reactive force on the tissue at the target implant region 106, e.g., to inhibit unintended rotation of first electrode 112 within the tissue.

All, substantially all, or a portion of housing 202 may function as an electrode 218, e.g., an anode, during pacing and/or sensing. In some examples, electrode 218 circumscribes a portion of housing 202 at or near proximal end 206. Electrode 218 can fully or partially circumscribe housing 202. FIG. 2A shows electrode 218 extending as a singular band around the outer perimeter of housing 202. Electrode 218 can also include multiple segments spaced a distance apart along a longitudinal axis 210 of housing 202 and/or around a perimeter of housing 202.

When housing 202 is formed from a conductive material, such as a titanium alloy, portions of housing 202 may be electrically insulated by a non-conductive material, such as a coating of parylene, polyurethane, silicone, epoxy or other biocompatible polymer, or other suitable material. For the portions of housing 202 without the non-conductive material, one or more discrete areas of housing 202 with conductive material can be exposed to define electrode 218.

When housing 202 is formed from a non-conductive material, such as a ceramic, glass or polymer material, an electrically-conductive coating or layer, such as a titanium, platinum, stainless steel, alloys thereof, a conductive material may be applied to one or more discrete areas of housing 202 to form electrode 218.

In some examples, electrode 218 is a component, such as a ring electrode, that is mounted or assembled onto housing 202. Electrode 218 may be electrically coupled to internal circuitry of device 104 via electrically-conductive housing 202 or an electrical conductor when housing 202 is a non-conductive material. In some examples, electrode 218 is located proximate to proximal end 206 of housing 202 and can be referred to as a proximal housing-based electrode. Electrode 218 can also be located at other positions along housing 202, e.g., located proximately to distal end 204 or at other positions along longitudinal axis 210.

In some examples, second electrode 114 or electrode 218 is paired with first electrode 112 for sensing ventricular signals and delivering ventricular pacing pulses. In some examples, second electrode 114 is paired with electrode 218 or first electrode 112 for sensing atrial signals and delivering pacing pulses to atrial tissue (e.g., to the atrial endocardium) in target implant region 106. In other words, electrode 218 is paired, at different times, with first electrode 112 and/or second electrode 114 for either ventricular or atrial functionality, respectively. In some examples, first and second electrodes 112 and 114 are paired with each other, with different polarities, for atrial and ventricular functionality.

In some examples, second electrode 114 is configured as an atrial cathode electrode for delivering pacing pulses to the atrial tissue, e.g., at target implant region 106 in combination with electrode 218. Second electrode 114 and electrode 218 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 218 as the return anode.

A distal end of first electrode 112 can be configured to rest within a ventricular myocardium of the patient, and second electrode 114 can be configured to contact an atrial endocardium of the patient without penetration of the atrial endocardium. Device 104 may include more or fewer electrodes than two electrodes. In some examples, device 104 includes one or more second electrodes 114 along housing distal end 204. For example, device 104 may include two or three electrodes configured for atrial functionality like second electrode 114, and the three electrodes may be substantially similar or different from one another. Spacing between a plurality of second electrodes 114 may be at an equal or unequal distance. Second electrode(s) 114 may be individually selectively coupled to sensing and/or pacing circuitry enclosed by housing 202 for use as an anode with first electrode 112 or as an atrial cathode electrode, or may be electrically common and not individually selectable. In some examples, in place of first electrode 112, device 104 includes a fixation element (not shown) of similar shape and mechanical, but without an electrically active region or electrode formed thereon or borne thereby; in such examples, electrically active region 216 can be positioned on a separate member and/or on the housing 202. In some examples, device 104 only includes first electrode 112 and electrode 218 and does not include any second electrodes 114.

In some examples device 104 includes one or more therapeutic substance dispensing devices, e.g., on face 205. The therapeutic substance dispensing devices may be configured to elute one or more steroids to tissue in proximity to the therapeutic substance dispensing devices over time. The steroid may mitigate inflammation of patient tissue resulting from interaction with device 104. In some examples, the therapeutic substance dispensing devices comprises one or more monolithic controlled release devices (MCRDs).

FIG. 2B is a perspective diagram illustrating an example side view of device 104 of FIG. 2A. FIG. 2C is a perspective diagram illustrating an example top view device 104 of FIG. 2A. As illustrated in FIGS. 2B and 2C, second electrode 114 may be disposed radially outward of first electrode 112 and may extend distally from face 205.

FIG. 2B illustrates second electrode 114 in an uncompressed configuration. As a clinician implants device 104 within tissue at tissue implantation region 106, the surface of the tissue may come into contact with a distal surface of second electrode 114. As the clinician further advances first electrode 112 into the tissue, the tissue may apply a force on second electrode 114 in a proximal direction along longitudinal axis 210, which may cause second electrode 114 to compress towards face 205.

An amount of displacement by second electrode 114 from the uncompressed configuration into an at least partially compressed configuration may affect an implantation depth of first electrode 112. In such examples, a maximum implantation depth of first electrode 112 may be up to a sum of a first distance between the distal end of first electrode 112 and a distal end of second electrode 114 in the uncompressed configuration (e.g., as measured along longitudinal axis 210) and a second distance corresponding to a maximum displacement of second electrode 114 along longitudinal axis 210 between the uncompressed configuration and a fully compressed configuration. The maximum implantation depth of first electrode 112 may vary based on the implantation angle of device 104 into the tissue. For example, device 104 may define a greater maximum implantation depth when implanted orthogonally to a surface of tissue compared to at an angle relative to the surface of the tissue.

FIG. 2C illustrates second electrode 114 as revolving fully around longitudinal axis 210, e.g., as to define a complete annular structure. In such examples, any portion of second electrode 114 may compress in response to a force applied on the respective portion of second electrode 114 (e.g., by the tissue). The compressibility of second electrode 114 around the entire circumference of second electrode 114 may increase a number of possible orientations of device 104 relative to the tissue during implantation of device 104, e.g., due to second electrode 114 being compressible at any position along the outer perimeter of device 104.

Second electrode 114 may define second electrically active region 220. Second electrically active region 220 may be a single continuous region extending substantially along the circumference of second electrode 114. In some examples, second electrically active region 220 includes two or more electrically active sub-regions separated by two or more electrically insulated sub-regions. Electrically active region 220 may be evenly distributed around longitudinal axis 210, e.g., to cause second electrode 114 to perform consistent in sensing and transmitting electrical signals independent of the orientation of device 104 to a surface of the tissue of target implantation region 106.

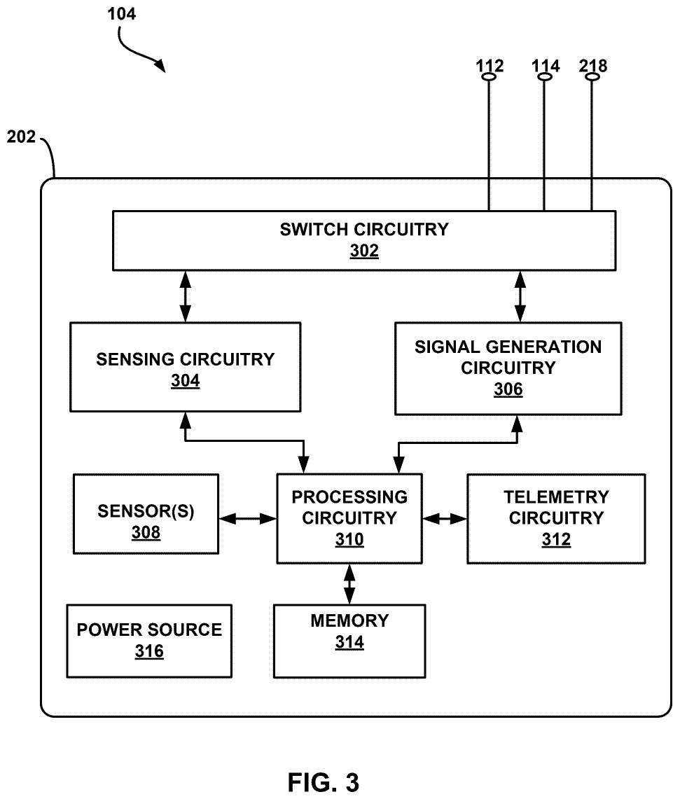

FIG. 3 is a functional block diagram illustrating an example configuration of device 104. As illustrated in FIG. 3, device 104 include electrodes 112 and 114, which may be configured as described with respect to FIGS. 1-2C. In the example shown in FIG. 3, device 104 includes switch circuitry 302, sensing circuitry 304, signal generation circuitry 306, sensor(s) 308, processing circuitry 310, telemetry circuitry 312, memory 314, and power source 316. The various circuitry may be, or include, programmable or fixed function circuitry configured to perform the functions attributed to respective circuitry. Memory 314 may store computer-readable instructions that, when executed by processing circuitry 310, cause device 104 to perform various functions. Memory 314 may be a storage device or other non-transitory medium. The components of device 104 illustrated in FIG. 3 may be housed within housing 202.

Signal generation circuitry 306 generates electrical stimulation signals, e.g., cardiac pacing pulses. Switch circuitry 302 is coupled to electrodes 112, 114, and 218 and may include one or more switch arrays, one or more multiplexers, one or more switches (e.g., a switch matrix or other collection of switches), one or more transistors, or other electrical circuitry. Switch circuitry 302 is configured to direct stimulation signals from signal generation circuitry 306 to a selected combination of electrodes 112, 114, and 218, having selected polarities, e.g., to selectively deliver pacing pulses to the RA, ventricles, or interventricular septum of heart 102. For example, in order to pace one or both of the ventricles, switch circuitry 302 may couple first electrode 112, which has penetrated to wall tissue of a ventricle or the intraventricular septum, to signal generation circuitry 306 as a cathode, and one or both of second electrode 114 or electrode 218 to signal generation circuitry 306 as an anode. As another example, in order to pace the RA, switch circuitry 302 may couple second electrode 114, which maintains contact with the RA endocardium, to signal generation circuitry 306 as a cathode, and one or both of first electrode 112 or electrode 218 to signal generation circuitry 306 as an anode.

Each of electrodes 112, 114, 218 may be coupled to switch circuitry 302 via a corresponding feedthrough assembly. In some examples, each feedthrough assembly is substantially straight (e.g., along longitudinal axis 210). In some examples, such as when distal end 204 of housing 202 is removable from housing 202 (e.g., when distal end 204 is a removable header), the feedthrough assemblies are offset to allow for removal of distal end 204. For example, when a header defining distal end 204 is configured to be removably secured to housing 202 (e.g., via a turn-lock mechanism), the feedthrough assemblies are offset from longitudinal axis 210 to allow the header to turn relative to housing 202.

Switch circuitry 302 may also selectively couple sensing circuitry 304 to selected combinations of electrodes 112, 114, and 218, e.g., to selectively sense the electrical activity of either the RA or ventricles of heart 102. Sensing circuitry 304 may include filters, amplifiers, analog-to-digital converters, or other circuitry configured to sense cardiac electrical signals via electrodes 112, 114, and/or 218. For example, switch circuitry 302 may couple each of first electrode 112 and second electrode 114 (in combination with electrode 218) to respective sensing channels provided by sensing circuitry 304 to respectively sense either ventricular or atrial cardiac electrical signals. In some examples, sensing circuitry 304 is configured to detect events, e.g., depolarizations, within the cardiac electrical signals, and provide indications thereof to processing circuitry 310. In this manner, processing circuitry 310 may determine the timing of atrial and ventricular depolarizations, and control the delivery of cardiac pacing, e.g., AV synchronized cardiac pacing, based thereon. Processing circuitry 310 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 310 herein may be embodied as firmware, hardware, software or any combination thereof.

Sensor(s) 308 may include one or more sensing elements that transduce patient physiological activity to an electrical signal to sense values of a respective patient parameter. Sensor(s) 308 may include one or more accelerometers, optical sensors, chemical sensors, temperature sensors, pressure sensors, or any other types of sensors. Sensor(s) 308 may output patient parameter values that may be used as feedback to control sensing and delivery of therapy by device 104.

Telemetry circuitry 312 supports wireless communication between device 104 and an external programmer (not shown in FIG. 3) or another computing device under the control of processing circuitry 310. Processing circuitry 310 of device 104 may receive, as updates to operational parameters from the computing device, and provide collected data, e.g., sensed heart activity or other patient parameters, via telemetry circuitry 312. Telemetry circuitry 312 may accomplish communication by radiofrequency (RF) communication techniques, e.g., via an antenna (not shown).

Power source 316 delivers operating power to various components of device 104. Power source 316 may include a rechargeable or non-rechargeable battery and a power generation circuit to produce the operating power. Recharging may be accomplished through proximal inductive interaction between an external charger and an inductive charging coil within device 104.

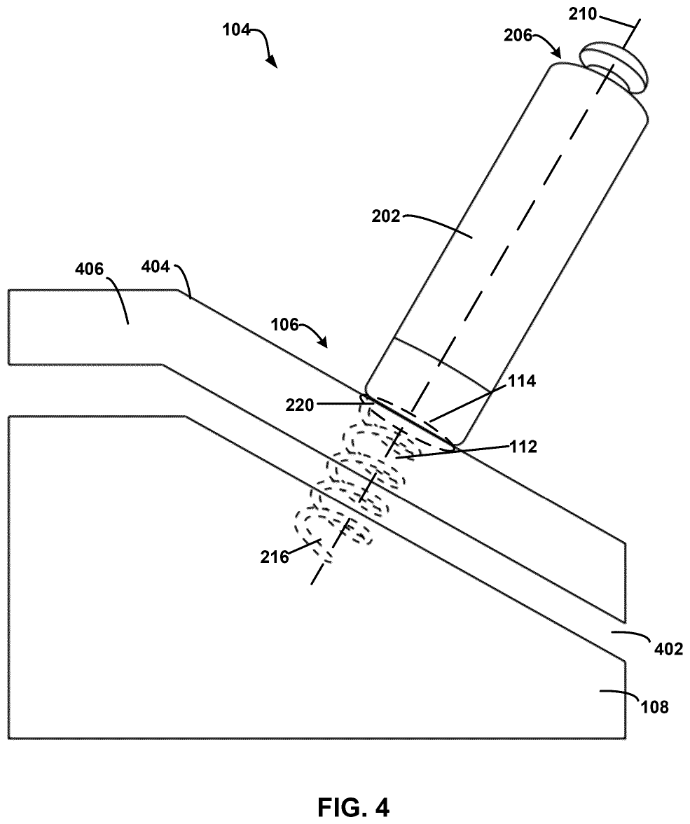

FIG. 4 is a conceptual diagram of an example device of any of FIGS. 1-3 implanted at a target implant site. First electrode 112 may be inserted (e.g., in a manner similar to rotating and advancing a threaded screw) such that tissue becomes engaged with the helix of first electrode 112. As first electrode 112 becomes engaged with tissue, first electrode 112 pierces into the tissue at target implant region 106 and advances through atrial myocardium 406 and central fibrous body 402 to position first electrically active region 216 in ventricular myocardium 108 as shown in FIG. 4. In some examples, first electrode 112 penetrates into the interventricular septum. In some examples, first electrode 112 does not perforate either of the ventricular endocardial or epicardial surface.

In some examples, manual pressure applied to the housing proximal end 206, e.g., via an advancement tool, provides the longitudinal force to pierce the cardiac tissue at target implant region 106. In some examples, actuation of an advancement tool rotates device 104 and first electrode 112 configured as a helix about longitudinal axis 210. The rotation of the helix about the longitudinal axis 210 advances first electrode 112 through atrial myocardium 406 and central fibrous body 402 to position first electrically active region 216 in ventricular myocardium 108 as shown in FIG. 4.

As first electrode 112 advances into the tissue, the distance between second electrode 114 and atrial endocardium 404 decreases until second electrode 114 contact, and may press against, the surface of atrial endocardium 404. Second electrode 114 may press against the surface of atrial endocardium 404 and compress the wall tissue. The compression of the wall tissue may prevent or inhibit rotation of device 104 due to movement of tissue of heart 102 (e.g., movement of ventricular myocardium 108, atrial myocardium 406, central fibrous body 402, or the like) or blood flow during cardiac function. The contact between second electrode 112 and the surface of atrial endocardium 404 may cause at least a portion of second electrode 112 to compress. Compression of second electrode 112 may increase a contact surface between atrial endocardium 404 and a second electrically active region 220 of second electrode 114. Compression of second electrode 112 may allow the clinician to further advance first electrode 112 into ventricular myocardium 108, e.g., to a target implant depth. Compression of second electrode 112 may facilitate successful penetration of first electrode 112 into ventricular myocardium 108 and successful fixation of first electrode 112 within ventricular myocardium 108, e.g., when the clinician implants device 104 at an angle from the surface of atrial endocardium 404. Retraction of second electrode 114 from the surface of atrial endocardium 404 may be prevented or inhibited by a spring force applied by second electrode 114 on atrial endocardium 404.

Target implant region 106 in some pacing applications is along atrial endocardium 604, substantially inferior to the AV node and bundle of His. For example, target implant region 106 may be at least partially within the Triangle of Koch of heart 102. First electrode 112 can have a length that penetrates through atrial 404 in target implant region 106, through the central fibrous body 402 and into ventricular myocardium 108 without perforating through the ventricular endocardial surface. In some examples, when the full length of first electrode 112 is fully advanced into target implant region 106, first electrically active region 216 rests within ventricular myocardium 108 and second electrode 114 is positioned in intimate contact with atrial endocardium 404. First electrode 112 may extend from housing distal end 204 approximately 3 mm to 12 mm in various examples. In some examples, first electrode 112 may extend a distance from distal end 204 by at least 3 mm, or at least 3 mm but in various examples. The diameter of an elongated body defining first electrode 112 may be 4 mm or less, e.g., may be 1 mm or less, may be 0.6 mm or less. An outer diameter of the helix or coil defined by first electrode 112 may be up to 6 mm (e.g., up to 4 mm).

For some locations for target implantation region 106, the surface of atrial endocardium 404 may be curved, may include protrusions, and/or may otherwise not define a substantially flat surface. The features on the surface of atrial endocardium 404 may require implantation of device 104 into the surface at an angle (e.g., where longitudinal axis 210 of device 104 is not orthogonal to the surface of atrial endocardium 404. In such examples, second electrode 112 may at least partially compress during implantation (e.g., as previously discussed above), which may increase fixation of device 104 to atrial endocardium 404 and increase the contact between second electrically active region 220 and the surface of atrial endocardium 404.

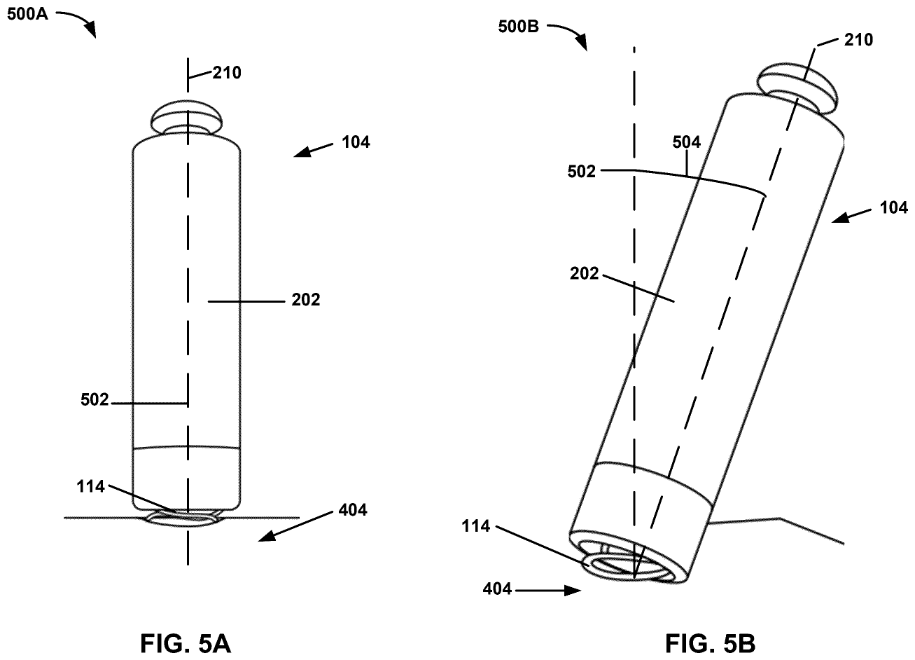

FIG. 5A is a conceptual diagram illustrating an orthogonal implantation orientation of device 104. FIG. 5B is a conceptual diagram illustrating an example angled implantation orientation of device 104. Device 104 is referred to herein as being implanted orthogonally into tissue within target implantation region 106 when a reference axis 502 orthogonal to the surface of the tissue aligns with longitudinal axis 210, e.g., as illustrated in FIG. 5A. Device 104 is referred to herein as being implanted at an angle into tissue within target implantation region 106 when longitudinal axis 210 is offset from reference axis 502 by implant angle 504 (alternatively referred to herein as “angle 504”). Angle 504 may be up to 45 degrees.

As illustrated in FIG. 5B, when device 104 is implanted into atrial endocardium 404 at angle 504, at least a portion of second electrode 114 may compress. The compression of second electrode 114 may increase the contact area between second electrically active region 220 (not shown in FIGS. 5A and 5B) and atrial endocardium 404, and/or increase fixation of device 104 to atrial endocardium 404. The increase range of angles 504 for implantation of device 104 may increase a number of possible implantation sites within heart 102 compared to other IMDs.

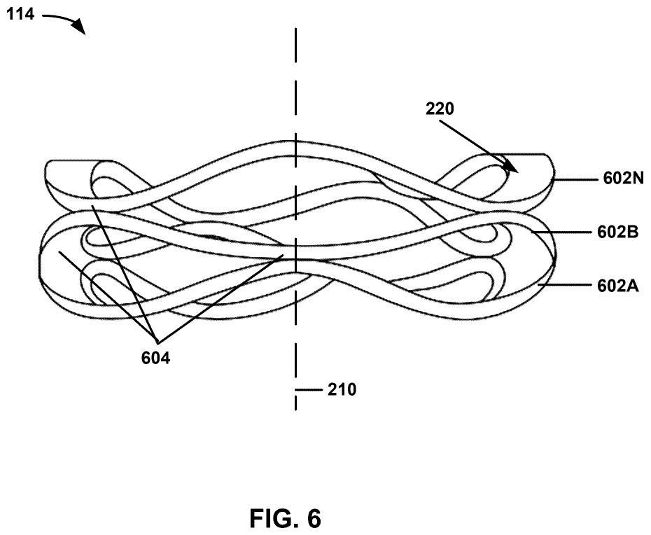

FIG. 6 is a conceptual diagram of an example compliant electrode (e.g., second electrode 114) of device 104. While FIG. 6 illustrates second electrode 114 as a wave spring electrode, second electrode 114 may include one or more other types of compliant electrodes, such as another type of spring electrode (e.g., a coil spring electrode). As illustrated in FIG. 6 second electrode 114 may include one or more spring layers (e.g., spring layers 602A-N, collectively referred to as “spring layers 602”). Each of spring layers 602 may define a wave structure around the perimeter of second electrode 114 and may be attached to one or more longitudinally adjacent spring layers 602 at node(s) 604.

A first spring layer 602A may define a proximal-most spring layer 602 along longitudinal axis 210. Spring layer 602A may be electrically connected to components of device 104 within housing 202 (e.g., to switch circuitry 302, sensing circuitry 304, signal generation circuitry 306). Spring layer 602A may be electrically connected to the components of device 104 via one or more wires, via a feedthrough assembly, or the like.

Each spring layer 602 may be permanently affixed to one or more longitudinally adjacent spring layers 602 at one or more nodes 604. Longitudinally adjacent spring layers 602 may be connected by one, two, three, or four or more nodes 604. Between two spring layers 602, node(s) 604 may be concentrated around a portion of the circumference of second electrode 114 or may be evenly distributed around the circumference of second electrode 114. even distribution of node(s) 604 around the circumference of second electrode 114 may cause spring layers 602 to define uniform stiffness and/or compliance around the outer perimeter of second electrode 114. Each spring layer 602 may define one or more undulations in a longitudinal direction (e.g., along longitudinal axis 210) along the circumference of spring layer 602. Node(s) 604 may be positioned at or around peaks and/or valleys in the one or more undulations in spring layer 602.

A manufacturing assembly may form each of node(s) 602 via welding longitudinally adjacent spring layers 602 together. At each node 602, one or more distal-most position on a first spring layer (e.g., first spring layer 602A) may each be affixed to a proximal-most position of one or more proximal-most position of a longitudinally distal spring layer (e.g., second spring layer 602B). Longitudinally adjacent spring layers 602 may be electrically connected at node(s) 604. In such examples, a distal-most spring layer (e.g., spring layer 602N) may be connected to a proximal-most spring layer (e.g., first spring layer 602A) via the intermediate node(s) 604 and spring layers 602 connecting the two. In such examples, electric signals (e.g., sensing signals, stimulation signals) may be transmitted between the components within housing 202 and second electrically active region 220 along a distal surface of a distal-most spring layer (e.g., spring layer 602N) through the spring layers 602 and node(s) 604 of second electrode 114.

The distal surface of spring layer 602N may define second electrically conductive region 220 of second electrode 114. A manufacturing assembly may coat at least a portion of the distal surface of spring layer 602N with an electrically insulating material and/or an electrically conductive material to define second electrically conductive region 220 (e.g., to control the surface area of second electrically conductive region 220). Second electrically conductive region 220 may be a single continuous region or may be two or more sub-regions. The two or more sub-regions may be evenly distributed around the perimeter of spring layer 602N, e.g., to allow substantially the entire circumference of second electrode 114 to sense and transmit electrical signals.

In some examples, second electrode 114 may be a coil spring electrode. The coil spring electrode may define a substantially flat and/or planar distal end. In such examples, the distal end of the coil spring electrode extends along a reference plane orthogonal to longitudinal axis 210 and may lay on top of a proximally adjacent coil wind of the coil spring electrode. The coil spring electrode may be shorter than first electrode 112 along longitudinal axis 210 and/or may be disposed radially outwards of first electrode 112.

While FIGS. 1-6 describe device 104 as configured to be implanted wholly within heart 102, the same structures and components described herein may be used to fix another implantable medical device within tissue of a patient. For example, an implantable medical device may include a fixation device similar to structure and/or function to helix and/or coil defined by first electrode 112 a compliant structure defined by second electrode 114. In such examples, the compliant structure of second electrode 114 may increase a number of angles a clinician may use to implant the fixation device. In such examples, the compliant structure of second electrode 114 may provide the clinician increased control on the implantation depth of the fixation device within the tissue.

Device 104, as described in FIGS. 1-6, may implanted within heart 102 and configured to deliver pacing signals to a single chamber of heart 102 or to two or more chambers of heart 102. In some examples, target implantation region 106 is located within a first chamber of heart 102 (e.g., RA of heart 102) to allow implantation of first electrode 112 towards a second chamber of heart 102 (e.g., LV of heart 102) separated from the first chamber. In such examples, device 104 may deliver pacing signals to cardiac tissue of the first chamber via second electrode 114 and deliver pacing signals to cardiac tissue of the second chamber via first electrode 112.

In some examples, target implantation region 106 is located within a chamber of heart 102 (e.g., RV of heart 102) and device 104 is configured to deliver pacing signals to cardiac tissue of the chamber (e.g., via one or more of first electrode 112, second electrode 114, or another therapy delivery element). For example, target implantation region 106 may be located within the RV and device 104 may be configured to deliver pacing signals to a left bundle branch (LBB) of heart 102. In such examples, the compliant structure of second electrode 114 may facilitate implantation of device 104 at target implantation region 106 at an angle offset from the tissue surface at target implantation region 106, e.g., thereby enabling implantation of device 104 at a location within heart 102 where there may be insufficient space for implantation of device 104 orthogonal to the tissue surface.

While first and second electrodes 112, 114 are primarily described herein as electrodes connected to device 104, a fixation device connected to an IMD may define fixation elements with substantially similar dimensions, features, and/or materials as first and second electrodes 112, 114. In such examples, the fixation elements (e.g., fixation helix, fixation elongated body, compliant spring) may be electrically isolated from IMD and may be configured to affix the IMD to tissue of the patient, e.g., without delivering medical therapy to the tissue. In such examples, target implantation region 106 may be disposed at one or more other locations within the patient, e.g., outside of heart 102.

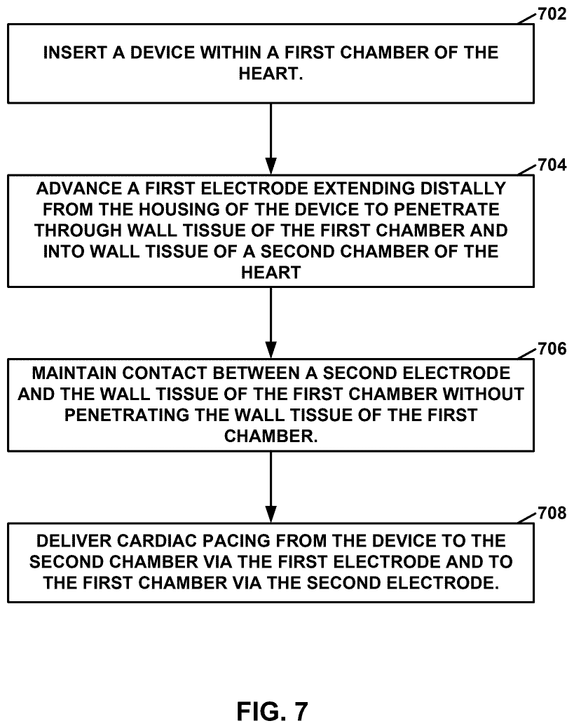

FIG. 7 is a flow diagram illustrating an example process for sensing a cardiac electrical signal and delivering cardiac pacing therapy to heart 102 of a patient via an device 104 of any of FIGS. 1-6. The technique of FIG. 7 will be described with concurrent reference to device 104 as illustrated in FIGS. 1-6, although a person having ordinary skill in the art will understand that the technique may be performed in reference to an implantable medical lead or other medical device.

A clinician may insert device 104 within a single first chamber of the heart 102 (702). The first chamber of heart 102 may be the right atrium, left atrium, the right ventricle, or the left ventricle. The clinician may insert device 104 into the first chamber via delivery tool connected to device 104 (e.g., connected to delivery tool interface member 208). The clinician may advance first electrode 112 extending distally from housing 202 of device 104 to penetrate through wall tissue of the first chamber and into wall tissue of a second chamber of heart 102 (704). In some examples, advancing first electrode 112 includes positioning a distal end of first electrode 112 (e.g., a first electrically active region 216) within a ventricular myocardium 108 of the patient. The clinician may advance first electrode 112 by rotating device 104 clockwise or counterclockwise within the first chamber, depending on how first electrode 112 is wound.

Depending on the surface features (e.g., surface curvature) of tissue within the target implantation region 106, the clinician may implant device 104 into the tissue orthogonally or at an angle. The clinician may position the distal end of first electrode 112 relative to the surface of the tissue such that longitudinal axis 210 is aligned with reference axis 502 (e.g., as illustrated in FIG. 5A) or such that an implantation angle 504 between longitudinal axis 210 and reference axis 502 is less than or equal to a threshold implantation angle (e.g., as illustrated in FIG. 5B). The clinician may then cause the distal end of first electrode 112 to penetrate the wall tissue of the first chamber and begin advancing first electrode 112 into the wall tissue (e.g., orthogonal to the surface of the wall tissue or at an angle offset from the surface of the wall tissue).

The clinician may cause device 104 to maintain contact between second electrode 114 and the wall tissue of the first chamber, without penetrating the wall tissue of the first chamber (706). As first electrode 112 is advanced into the wall tissue, at least a portion of second electrode 114 may be at least partially compressed, e.g., due to a compressive force acting on second electrode 114 from the wall tissue and from housing 202 of device 104. The at least partial compression may cause second electrode 114 to maintain full contact between second electrically active region 220 of second electrode 114 and the wall tissue, e.g., even where longitudinal axis 210 is offset from reference axis 502.

The clinician may continue to advance first electrode 112 into the tissue until at least a portion of second electrode 114 is fully compressed. The compliance of second electrode 114, and by extension a maximum displacement of second electrode 114 from the uncompressed configuration to the fully compressed configuration, may depend on the tissue within target implantation region 106 and the target implantation depth. When second electrode 114 is fully compressed, first electrically active region 216 of first electrode 112 may be positioned within the wall tissue at greater than or equal to the target implantation depth. Once device 104 is implanted within the wall tissue, second electrode 114 and/or one or more fixation features described herein may interface with the wall tissue to inhibit unintended movement of first electrode 112 out of the wall tissue.

The clinician may deliver cardiac pacing from device 104 to the second chamber via first electrode 112 and to the first chamber via second electrode 114 (708). Device 104 may deliver cardiac pacing to the first chamber and/or the second chamber via first electrode 112, second electrode 114, and/or one or more other electrodes of device 104 (e.g., electrode 218).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

This disclosure describes each of the following examples.

Example 1: a device comprising: an elongated housing extending from a proximal end to a distal end along a longitudinal axis, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode extends wholly around the longitudinal axis, wherein the second electrode is configured to at least partially deform to contact wall tissue of the chamber without penetrating the wall tissue.

Example 2: the device of example 1, wherein the second electrode comprises a wave spring electrode, and wherein the wave spring electrode is configured to at least partially elastically deform to contact the wall tissue of the chamber.

Example 3: the device of example 2, wherein the wave spring electrode comprises at least one spring layer.

Example 4: the device of any of examples 2 or 3, wherein the wave spring electrode defines an annular structure.

Example 5: the device of any of examples 2-4, wherein the wave spring electrode defines one or more undulations extending along the longitudinal axis around a circumference of the wave spring electrode

Example 6: the device of any of examples 2-5, wherein the wave spring electrode comprises one or more of Platinum Iridium, a Platinum Iridium-clad metallic alloy, or Tantalum Tungsten.

Example 7: the device of any of examples 2-6, wherein the wave spring electrode is configured to at least partially deform along a circumference of the wave spring electrode to allow the first electrode to penetrate the wall tissue when the longitudinal axis is not orthogonal to a surface of the wall tissue.

Example 8: the device of example 1, wherein the second electrode comprises a coil spring electrode, and wherein the coil spring electrode is configured to at least partially elastically deform to contact the wall tissue of the chamber.

Example 9: the device of example 8, wherein the coil spring electrode defines a substantially flat distal end, wherein the distal end of the coil spring electrode extends along a reference plane, the reference plane being substantially orthogonal to the longitudinal axis.

Example 10: the device of any of examples 8 or 9, wherein the coil spring electrode is shorter than the first electrode along the longitudinal axis.

Example 11: the device of any of example 1-10, wherein the second electrode defines a compliance, wherein the compliance is based at least in part on a first spring constant of the second electrode and a second spring constant of the wall tissue of the chamber.

Example 12: the device of any of examples 1-11, wherein the second electrode defines one or more electrically conductive regions, and wherein when the second electrode is at least partially deformed to contact the wall tissue of the chamber, the one or more electrically conductive regions are placed in contact with the wall tissue.

Example 13: the device of example 12, wherein the one or more electrically conductive regions comprises at least two electrically conductive regions, each electrically conductive region of the at least two electrically conductive regions being separated along the circumference of the second electrode from another of the at least two electrically conductive regions by an electrically insulated region.

Example 14: the device of any of examples 1-13, wherein the chamber of the heart comprises a first chamber of the heart, and wherein the helix is configured to penetrate into wall tissue of a second chamber of the heart that is separated from the first chamber of the heart.

Example 15: the device of example 14, wherein the first chamber comprises an atrium of the heart, and wherein the second chamber comprises a ventricle of the heart.

Example 16: the device of any of examples 1-15, wherein the second electrode is configured to, when at least partially deformed, inhibit unintended rotation of the elongated body within the wall tissue.

Example 17: a fixation device comprising: an elongated body extending distally from a distal end of an implantable medical device along a longitudinal axis, the elongated body comprising: a proximal end located at the distal end of the implantable medical device, and a helix extending distally from the proximal end along the longitudinal axis and defining one or more coils, wherein a distal end of the helix is configured to penetrate into tissue of a patient; and a spring disposed on the distal end of the implantable medical device, wherein the spring extends wholly around the longitudinal axis, and wherein the spring is configured to at least partially deform to contact the tissue without penetrating the tissue to inhibit unintended rotation of the elongated body within the tissue.

Example 18: the fixation device of example 17, wherein the spring comprises a wave spring, and wherein the wave spring is configured to at least partially elastically deform to contact the tissue.

Example 19: the fixation device of example 18, wherein the wave spring comprises at least one spring layer.

Example 20: the fixation device of any of examples 18 or 19, wherein the wave spring defines an annular structure.

Example 21: the fixation device of any of examples 18-20, wherein the wave spring defines one or more undulations extending along the longitudinal axis around a circumference of the wave spring.

Example 22: the fixation device of any of examples 18-21, wherein the wave spring comprises one or more of Platinum Iridium, a Platinum Iridium-clad metallic alloy, or Tantalum Tungsten.

Example 23: the fixation device of any of examples 18-22, wherein the wave spring is configured to at least partially deform along a circumference of the wave spring to allow the helix to penetrate the tissue when the longitudinal axis is not orthogonal to a surface of the tissue.

Example 24: the fixation device of example 17, wherein the spring comprises a coil spring, and wherein the coil spring is configured to at least partially elastically deform to contact the tissue.

Example 25: the fixation device of example 24, wherein the coil spring defines a substantially flat distal end, wherein the distal end of the coil spring extends along a reference plane, the reference plane being substantially orthogonal to the longitudinal axis.

Example 26: the fixation device of any of examples 24 or 25, wherein the coil spring is shorter than the helix along the longitudinal axis.

Example 27: the fixation device of example 26, wherein a first distal end of the helix is more distal from the distal end of the implantable medical device along the longitudinal axis than a second distal end of the coil spring.

Example 28: the fixation device of any of examples 17-26, wherein the spring defines a compliance, wherein the compliance is based at least in part on a first spring constant of the spring and a second spring constant of the tissue.

Example 29: a method comprising: inserting a device into a chamber of a heart, the device comprising: an elongated housing extending from a proximal end to a distal end along a longitudinal axis, a first electrode extending distally from the distal end of the elongated housing, the first electrode comprising an elongated body defining a helix, and a second electrode disposed on the distal end of the elongated housing, wherein the second electrode extends wholly around the longitudinal axis; advancing the first electrode to penetrate wall tissue of the chamber, wherein the second electrode is configured to at least partially deform as the first electrode penetrates the wall tissue; and delivering cardiac pacing from the device to the wall tissue via at least one of the first electrode or the second electrode.

Example 30: the method of example 29, wherein the second electrode comprises a wave spring electrode.

Example 31: the method of example 30, wherein the wave spring electrode comprises at least one spring layer.

Example 32: the method of any of examples 30 or 31, wherein the wave spring electrode defines an annular structure.

Example 33: the method of any of examples 30-32, wherein the wave spring electrode defines one or more undulations extending along the longitudinal axis around a circumference of the wave spring electrode

Example 34: the method of example 29, wherein the second electrode comprises a coil spring electrode.

Example 35: the method of example 34, wherein the coil spring electrode defines a substantially flat distal end, wherein the distal end of the coil spring electrode extends along a reference plane, the reference plane being substantially orthogonal to the longitudinal axis.

Example 36: the method of any of examples 34 or 35, wherein the coil spring electrode is shorter than the first electrode along the longitudinal axis.

Example 37: the method of any of examples 29-36, wherein advancing the first electrode to penetrate the wall tissue of the chamber comprises advancing the first electrode along a reference axis to penetrate the wall tissue, wherein the reference axis is not orthogonal to a surface of the wall tissue, and wherein advancing the first electrode along the reference axis causes a first portion of the second electrode to compress along the reference axis and a second portion of the second electrode to expand along the reference axis, the second portion being different from the first portion.

Example 38: the method of example 37, wherein the first and second portions are on opposite sides of the second electrode.

Example 39: the method of any of examples 29-38, wherein the chamber of the heart comprises a first chamber of the heart, wherein advancing the first electrode to penetrate the wall tissue of the chamber comprises advancing the first electrode through wall tissue of the first chamber of the heart and into wall tissue of a second chamber of the heart that is separated from the first chamber, and wherein delivering cardiac pacing from the device to the wall tissue via at least one of the first electrode or the second electrode comprises: delivering a first cardiac pacing from the device to the wall tissue of the second chamber of the heart via the first electrode; and delivering a second cardiac pacing from the device to the wall tissue of the first chamber of the heart via the second electrode.

Example 40: the method of example 39, wherein the first chamber comprises an atrium of the heart, and wherein the second chamber comprises a ventricle of the heart.

Various examples have been described. These and other examples are within the scope of the following claims.

What is claimed is:

1. A device comprising:
an elongated housing extending from a proximal end to a distal end along a longitudinal axis, the elongated housing being configured to be implanted wholly within a chamber of a heart;
a first electrode extending distally from the distal end of the elongated housing, the first electrode comprising an elongated body defining a helix;
a second electrode disposed on the distal end of the elongated housing, wherein the second electrode extends wholly around the longitudinal axis, wherein the second electrode is configured to at least partially deform to contact wall tissue of the chamber without penetrating the wall tissue.

2. The device of claim 1, wherein the second electrode comprises a wave spring electrode, and wherein the wave spring electrode is configured to at least partially elastically deform to contact the wall tissue of the chamber.

3. The device of claim 2, wherein the wave spring electrode comprises at least one spring layer.

4. The device of claim 2, wherein the wave spring electrode defines an annular structure.

5. The device of claim 2, wherein the wave spring electrode defines one or more undulations extending along the longitudinal axis around a circumference of the wave spring electrode

6. The device of claim 2, wherein the wave spring electrode comprises one or more of Platinum Iridium, a Platinum Iridium-clad metallic alloy, or Tantalum Tungsten.

7. The device of claim 2, wherein the wave spring electrode is configured to at least partially deform along a circumference of the wave spring electrode to allow the first electrode to penetrate the wall tissue when the longitudinal axis is not orthogonal to a surface of the wall tissue.

8. The device of claim 1, wherein the second electrode comprises a coil spring electrode, and wherein the coil spring electrode is configured to at least partially elastically deform to contact the wall tissue of the chamber.

9. The device of claim 8, wherein the coil spring electrode defines a substantially flat distal end, wherein the distal end of the coil spring electrode extends along a reference plane, the reference plane being substantially orthogonal to the longitudinal axis.

10. The device of claim 1, wherein the second electrode defines a compliance, wherein the compliance is based at least in part on a first spring constant of the second electrode and a second spring constant of the wall tissue of the chamber.

11. The device of claim 1, wherein the second electrode defines one or more electrically conductive regions, and wherein when the second electrode is at least partially deformed to contact the wall tissue of the chamber, the one or more electrically conductive regions are placed in contact with the wall tissue.

12. The device of claim 11, wherein the one or more electrically conductive regions comprises at least two electrically conductive regions, each electrically conductive region of the at least two electrically conductive regions being separated along the circumference of the second electrode from another of the at least two electrically conductive regions by an electrically insulated region.

13. The device of claim 1, wherein the second electrode is configured to, when at least partially deformed, inhibit unintended rotation of the elongated body within the wall tissue.

14. A fixation device comprising:
an elongated body extending distally from a distal end of an implantable medical device along a longitudinal axis, the elongated body comprising:
a proximal end located at the distal end of the implantable medical device, and
a helix extending distally from the proximal end along the longitudinal axis and defining one or more coils, wherein a distal end of the helix is configured to penetrate into tissue of a patient; and
a spring disposed on the distal end of the implantable medical device, wherein the spring extends wholly around the longitudinal axis, and wherein the spring is configured to at least partially deform to contact the tissue without penetrating the tissue to inhibit unintended rotation of the elongated body within the tissue.

15. The fixation device of claim 14, wherein the spring comprises a wave spring, and wherein the wave spring is configured to at least partially elastically deform to contact the tissue.

16. The fixation device of claim 15, wherein the wave spring defines one or more undulations extending along the longitudinal axis around a circumference of the wave spring.

17. The fixation device of claim 15, wherein the wave spring is configured to at least partially deform along a circumference of the wave spring to allow the helix to penetrate the tissue when the longitudinal axis is not orthogonal to a surface of the tissue.

18. The fixation device of claim 14, wherein the spring comprises a coil spring, and wherein the coil spring is configured to at least partially elastically deform to contact the tissue.

19. A method comprising:
inserting a device into a chamber of a heart, the device comprising:
an elongated housing extending from a proximal end to a distal end along a longitudinal axis,
a first electrode extending distally from the distal end of the elongated housing, the first electrode comprising an elongated body defining a helix, and
a second electrode disposed on the distal end of the elongated housing, wherein the second electrode extends wholly around the longitudinal axis;
advancing the first electrode to penetrate wall tissue of the chamber, wherein the second electrode is configured to at least partially deform as the first electrode penetrates the wall tissue; and
delivering cardiac pacing from the device to the wall tissue via at least one of the first electrode or the second electrode.

20. The method of claim 19, wherein the second electrode comprises a wave spring electrode.
